(19) United States
(12) Patent Application Publication
(10) Pub. No.: US 2021/0233613 A1
(43) Pub. Date: Jul. 29, 2021

(54) METHOD FOR CREATION OF A CONSISTENT REFERENCE BASIS FOR GENOMIC COMPARISONS

(71) Applicant: KONINKLIJKE PHILIPS N.V., EINDHOVEN (NL)

(72) Inventor: Helen Cecile van Aggelen, Somerville, MA (US)

(21) Appl. No.: 17/051,906

(22) PCT Filed: Jun. 11, 2019
(86) PCT No.: PCT/EP2019/065088

§ 371 Date: Oct. 30, 2020

Related U.S. Application Data
(60) Provisional application No. 62/684,323, filed on Jun. 13, 2018.

(51) Int. Cl. G16B 30/10

(52) U.S. Cl. CPC G16B 30/10

(57) ABSTRACT

A method (100) for generating a genome reference using a genome reference system (300), comprising: (i) receiving (110), by the system, sequencing data for a plurality of genomes, the sequencing data generated from a plurality of genomes obtained from a single species; (ii) selecting (120), by a processor (320), sequencing data from one of the plurality of genomes; (iii) aligning (130) the selected sequencing data from the selected genome, comprising a plurality of k-mers, with each of the plurality of genomes; (iv) determining (140), based on the alignment, a frequency of each of the plurality of k-mers within the plurality of genomes; (v) selecting (160), based on the frequency determination, one or more base positions within the plurality of k-mers that exceed a predetermined frequency threshold; (vi) assigning (170) the selected base positions to a genome reference; and (vii) storing (180) the genome reference in a data structure (326, 360).

### FIELD OF THE DISCLOSURE

The present disclosure is directed generally to methods and systems for generating a genome reference.

### BACKGROUND

Genomic analysis has made it possible to quickly and accurate determine the identity of pathogens, and is increasingly being applied in clinical settings. As the amount of sequencing data available for analysis continues to grow, methods for rapid comparison between genomes are needed to detect quickly identify infectious disease threats and emerging new pathogens, to monitor outbreaks, and for many other uses.

A basis for comparison is required when trying to identify the genomic source of sequenced samples. This may be a whole reference genome to which sample read data is aligned and variant-called. The genomic distance between samples can then be determined as the number of base pairs or variants that are different between the consensus sequences. However, this approach can produce highly variable and inconsistent distances. For example, certain regions of the genome can be highly variable and bias the distance metric, and some regions of the reference may be missing in the sample, among other issues.

A straightforward approach to compare samples relative to a common reference genome is to consider only those base pairs in the reference genome that are well determined in all samples. To produce consistent genomic distances, comparison between genomes is often done relative to a core genome which consists of genes that are present in all reference genomes considered. Only genomic differences that fall into the core genome regions are then considered in the calculation of the genomic distance.

The drawback of this approach is that the selection of reference base pairs can change upon addition of new samples, which leads to inconsistent genomic distances. Such methodologies, while reliable in performing retrospective analyses, cannot be used prospectively. With dynamic studies in which genomes are iteratively added to a previously analyzed dataset, consensus loci observed across genomes will continue to shrink, resulting in shifting genomic distances over time. This approach is therefore unsuitable for, among many other applications and uses, a clinical product aimed at tracking infections over time.

### SUMMARY OF THE DISCLOSURE

There is a continued need for methods and systems that generate a genome reference which produces to consistent genomic distances.

The present disclosure is directed to inventive methods and systems for generating a genome reference. Various embodiments and implementations herein are directed to a system that receives sequencing data for a plurality of genomes obtained from a single species for which the genome reference will be generated. One of the genomes is selected, and the k-mers from the sequencing data of the selected genome are aligned with the other genomes in the set. The frequency of each of the k-mers within the other genomes in the set is determined by the alignment, and base positions within the k-mers that exceed a predetermined threshold are assigned to a genome reference. The generated genome reference is stored in a data structure and is configured to be used to compare to sequencing data from a sample genome of the same species.

Generally in one aspect, is a method for generating a genome reference using a genome reference system. The method includes: (i) receiving, by the system, sequencing data for a plurality of genomes, the sequencing data generated from a plurality of genomes obtained from a single species; (ii) selecting, by a processor of the system, sequencing data from one of the plurality of genomes; (iii) aligning, by the processor, the selected sequencing data from the selected genome, comprising a plurality of k-mers, with each of the plurality of genomes; (iv) determining, by the processor, based on the alignment, a frequency of each of the plurality of k-mers within the plurality of genomes; (v) selecting, by the processor based on the frequency determination, one or more base positions within the plurality of k-mers that exceed a predetermined frequency threshold; (vi) assigning, by the processor, the selected base positions to a genome reference; and (v) storing the genome reference in a data structure.

According to an embodiment, the sequencing data comprises whole genome sequencing data. According to an embodiment, the sequencing data comprises genome assemblies.

According to an embodiment, the method further includes the step of identifying base positions within the plurality of k-mers using a transformation function. According to an embodiment, the transformation function is a running maximum or a running average.

According to an embodiment, the step of aligning the selected sequencing data from the selected genome with each of the plurality of genomes requires identity between the sequencing data and a region of the one of the plurality of genomes.

According to an embodiment, the step of aligning the selected sequencing data from the selected genome with each of the plurality of genomes allows a predetermined level of mismatch between the sequencing data and a region of the one of the plurality of genomes.

According to an embodiment, the predetermined threshold is 0.9.

According to an embodiment, the method further includes the step of comparing a sample to the genome reference.

According to an embodiment, receiving sequencing data for a plurality of genomes comprises generating sequencing data using a sequencing platform.

According to an embodiment, the method further includes computing coverage metrics for a plurality of base positions across a plurality of sequence samples obtained from a single species; and comparing the coverage metrics for the plurality of base positions to a predetermined coverage threshold to identify a set of highly covered base positions, wherein selecting the one or more base positions includes selecting one or more base positions within the plurality of k-mers that both: exceed the predetermined frequency threshold, and are associated with a coverage metric of the coverage metrics that exceed the predetermined coverage threshold.

In one aspect is a system for generating a genome reference using a genome reference system. The system includes a processor configured to: (i) receive sequencing data for a plurality of genomes, the sequencing data generated from a plurality of genomes obtained from a single species; (ii) select sequencing data from one of the plurality of genomes; (iii) align the selected sequencing data from the selected genome, comprising a plurality of k-mers, with each of the plurality of genomes; (iv) determined, based on the alignment, a frequency of each of the plurality of k-mers within the plurality of genomes; (v) select, based on the frequency determination, one or more base positions within the plurality of k-mers that exceed a predetermined frequency threshold; and (vi) assign the selected base positions to a genome reference; and a data structure configured to store the genome reference.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for generating a genome reference, in accordance with an embodiment.

FIG. 2 is a flowchart of a method for generating a genome reference, in accordance with an embodiment.

FIG. 3 is a schematic representation of a system for generating a genome reference, in accordance with an embodiment.

### DETAILED DESCRIPTION OF EMBODIMENTS

The present disclosure describes various embodiments of a system and method for generating a genome reference for a species using sequencing data from a plurality of sample genomes of that species. More generally, Applicant has recognized and appreciated that it would be beneficial to provide a genome reference for a species that produces consistent genomic distances as new samples are compared. The system, which may optionally comprise a sequencing platform, generates or receives sequencing data, such as whole genome data and/or genome assemblies, for a plurality of genomes obtained from a single species for which the genome reference will be generated. One of the genomes is selected, and the k-mers from the sequencing data of the selected genome are aligned with the other genomes in the set. The frequency of each of the k-mers within the other genomes in the set is determined by the alignment, and base positions within the k-mers that exceed a predetermined threshold are assigned to a genome reference. The generated genome reference is stored in a data structure and is configured to be used to compare to sequencing data from a sample genome of the same species.

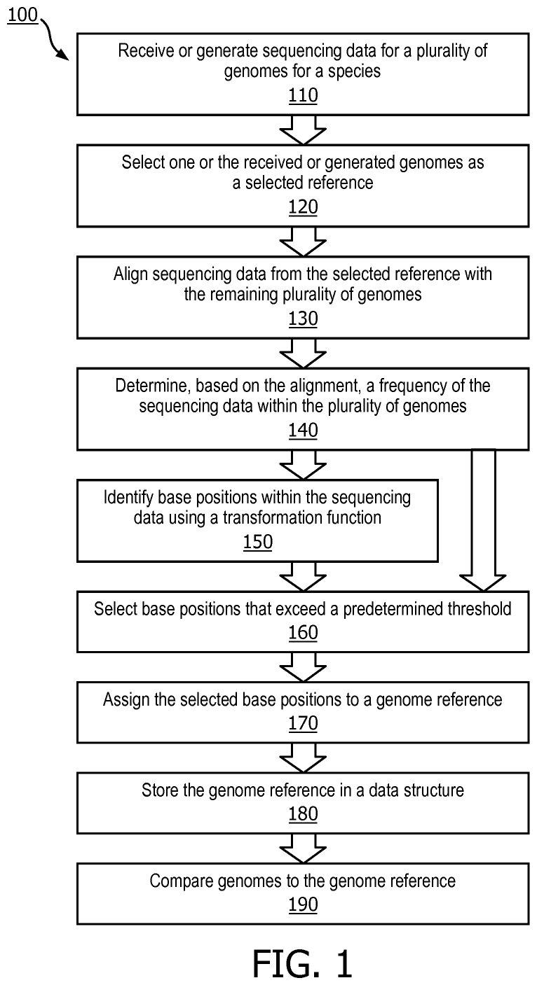

Referring to FIG. 1, in one embodiment, is a flowchart of a method 100 for generating a genome reference using a genome reference system. The genome reference system may be any of the systems described or otherwise envisioned herein, and may comprise any of the components described or otherwise envisioned herein.

At step 110 of the method, the genome reference system generates and/or receives sequencing data for a plurality of genomes. Each of the plurality of genomes is obtained from a single species, or samples believed to comprise a single species or comprise mostly a single species. As non-limiting examples, the species can be pathogenic, such as K. pneumoniae, S. aureus, and/or P. aeruginosa, non-pathogenic, or of unknown pathogenicity and/or origin, among many other types or varieties of species.

It is recognized that there is no limitation to the source of the species for the generated genome reference. For example, the plurality of genomes may comprise a population or sub-population of genomes generated or obtained according to many different criteria and/or methodologies. According to an embodiment, the genomes are generated or obtained from samples collected from a single location, several locations, or many locations. According to an embodiment, the genomes are generated over a plurality of time points. For example, the genomes may be generated or obtained from samples collected from one or more than one location over two or more points in time. The two or more points in time may be selected based on a wide variety of different criteria and/or methodologies. As another embodiment, the genomes are generated or obtained from samples collected from a single location, several locations, or many locations over two or more points in time.

According to an embodiment, the genome reference system comprises a sequencing platform configured to obtain one or more genomes for the plurality of genomes. The sequencing platform can be any sequencing platform, including but not limited to any systems described or otherwise envisioned herein. For example, the sequencing platform can be a real-time single-molecule sequencing platform, such as a pore-based sequencing platform, although many other sequencing platforms are possible. The sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner.

According to an embodiment, the genome reference system receives the sequencing data for one or more of the plurality of genomes. For example, the genome reference system may be in communication or otherwise receive data from a genome database comprising one or more genomes for the target species. For example, the genome database may be a public database comprising many genomes of the target species, and/or may be a private or institutional database comprising one or more genomes of the target species. As just one non-limiting example, the sequencing data may be obtained from or otherwise received from reference sequences in the NCBI RefSeq, among many other databases. The generated and/or received sequencing data may be comprise a plurality of k-mers for each of the plurality of genomes for a species.

The generated and/or received sequencing data may be NCBI RefSeq may be stored in a local or remote database for use by the genome reference system. For example, the genome reference system may comprise a database to store the sequencing data for the plurality of genomes, and/or may be in communication with a database storing the sequencing data. These databases may be located with the genome reference system or may be located remote from the genome reference system, such as in cloud storage and/or other remote storage.

The generated and/or received sequencing data may be complete genomes, or may be partial genomes. For example, the generated and/or received sequencing data may be assemblies, whole genome constructs, incomplete genomes, partial genomes, and/or any other sequencing data. The generated and/or received sequencing data may comprise any number of genomes. For example, the number of genomes may be limited or may be expansive based on the species being analyzed. As a non-limiting example, the number of genomes may be approximately 1,000, although the number of genomes may be may be any number smaller or greater than 1,000.

At step 120 of the method, one of the plurality of genomes received or generated by the genome reference system is selected to be a selected reference. The selected reference can be any of the genomes received or generated by the genome reference system. The selected reference may be randomly selected, or selected based upon one or more criteria, including completeness of the sample, the quality of the sequencing data, and/or any other criterion. Selection of the selected reference may comprise, for example, associating a stored version of copy of the genome with an identifier in memory, or extracting the selected reference from a database, and/or otherwise preparing the selected genome for downstream steps of the method. For example, the sequencing data comprising a plurality of k-mers for the selected genome can be located within a database, and can be extracted, copied, or otherwise prepared for analysis.

At step 130 of the method, the sequencing data from the selected reference is aligned with the remainder of the genomes in the plurality of genomes. For example, the sequencing data for the selected genome may comprise a plurality of k-mers that are aligned with each of the other genomes for the species in the database or otherwise obtained or generated by the genome reference system. The sequencing data from the selected reference may be aligned with the remainder of the genomes using any method of alignment, including but not limited to known alignment algorithms or methods. According to an embodiment, the system may compare each of the plurality of k-mers to the genomes in the plurality of genomes one by one in turn, or may align all of the plurality of k-mers with the genomes in the plurality of genomes at once, sequentially, or in another manner.

According to an embodiment, the genome reference system or method requires identity between the sequencing data and a region of the genome to which the sequencing data is being aligned. Thus, if the genome comprises a variant not found in a k-mer, for example, the k-mer will not be aligned. According to another embodiment, the genome reference system or method allows for some mismatch between the sequencing data and a region of the genome to which the sequencing data is being aligned. Thus, if the genome comprises a number of variants at or below the mismatch threshold, which may be one or any other amount, the k-mer will be identified as aligning with the genome.

According to an embodiment, the genome reference system preferentially aligns long reads from the selected reference with the remainder of the genomes in the plurality of genomes. The length of a read required to be considered a long read and thus preferentially aligned can be defined by a user, by the system, by a machine learning algorithm, and by a variety of other mechanisms. According to an embodiment, preferentially aligning long reads may accelerate the analysis process and/or other processes of the genome reference system.

At step 140 of the method, the genome reference system uses the alignment information to determine a frequency of the sequencing data within the plurality of genomes. For example, in step 130 of the method a k-mer is compared to each of the genomes in the plurality of genomes during the alignment step. A k-mer may align with all the genomes (100%), with none of the genomes (0%), or with a percentage of the genomes greater than 0% and less than 100%. The genome reference system tracks or records the alignment frequency for each piece of sequencing data for the selected reference, such as a k-mer, for example using a counter or any other tracking or recording method. Thus, the genome reference system comprises an identification of alignment frequency for the sequencing data, such as for the plurality of k-mers.

According to an embodiment, the sequencing data is associated with frequency information in memory, such as a table. For example, each of the plurality of k-mers of for the selected reference may be associated in a table or other data structure with the frequency for that respective k-mer.

At optional step 150 of the method, one or more base positions with the sequencing data is identified using a transformation function. To measure the frequency of a single base within the sequencing data, which may comprise overlapping k-mers or other overlapping sequencing data, a transformation function is applied to the data. For example, the system may perform a running maximum, average, or another function of the relative counts as a frequency measure. As just one non-limiting example, a running maximum of the data may be performed in windows of k=3 base pairs, starting 2 base pairs ahead of each position. For example, a running maximum can be taken over a window of k positions such that each position p is mapped to the maximum of the relative k-mer frequency over the window [p−k+1, p]. This and other transformation functions are possible. For example, the frequency measure can also be computed by multiple alignment of the reference genomes.

The transformation function generates a plurality of base positions within the sequencing data of the selected reference, which can be stored in memory, a database, or otherwise stored and/or utilized for further steps of the analysis. According to an embodiment, each of the base positions is associated with frequency information in memory, such as a table. For example, each of the base positions in the sequencing data may be associated in a table or other data structure with the frequency for that respective base position.

At step 160 of the method, the genome reference system selects one or more base positions of the selected reference that exceeds a predetermined frequency threshold. For example, each of the base positions of the selected reference may be associated with a frequency determined in one or more of the previous steps of the method. This association may be in memory, a database, or any other data structure. The genome reference system may be configured or designed to select base positions that meet or exceed a predetermined threshold. The predetermined threshold may be a user-entered variable, a variable determined by trial and error, a variable determined by machine learning, or a variable determined by any other method. As just one non-limiting example, the predetermined threshold may be 90%, although any number above or below 90% may be suitable. Thus, the system includes position p in the genome reference if the conservation score exceeds the 90% threshold. As another non-limiting example, the predetermined threshold may be 95%, although any number above or below 95% may be suitable. According to one embodiment, the predetermined threshold may be much lower to aim for regions that have greater variability. For example, as one non-limiting example, the predetermined threshold may be between 40 and 60%, inclusive, to capture greater variability, although thresholds greater or smaller than 40-60% may be utilize variability found among the genomes in the data set.

According to an embodiment, all base positions and/or sequencing data that exceed the predetermined threshold may be selected. As another option, only some base positions and/or sequencing data that exceed the predetermined threshold may be selected. For example, some regions of a genome may be identified for exclusion and/or inclusion relative to the selection of base positions and/or sequencing data. According to another embodiment, the predetermined threshold may vary along the genome. For example, base positions and/or sequencing data from some regions of the genome may be subjected to a first threshold, while base positions and/or sequencing data from other regions of the genome may be subjected to a second threshold, where the first and second thresholds are different. For example, the first threshold may be higher than the second threshold, or vice versa.

According to an embodiment, the genome reference system may be configured or designed to utilize two or more different thresholds to select base positions. For example, the genome reference system may apply a first threshold to a first set of specific regions of the genome, and may apply a second threshold to a second set of specific regions of the genome, the first set of specific regions different from the second set of specific regions. A plurality of different thresholds and regions are possible. As just one example, the genome reference system may utilize a lower threshold—relative to the threshold used for other regions of the genome—for regions of hyper-variability in the genome. These hyper-variable regions may be identified by the genome reference system, defined by a user, or provided by other mechanisms. According to another example, the genome reference system may utilize a higher threshold-relative to the threshold used for other regions of the genome—for highly conserved regions of the genome. Many other variations are possible.

In some alternative embodiments, the core genome may be constructed of regions that are both highly conserved (as described above) and that have sufficiently high coverage. For example, in some embodiments, the method 100 may include additional steps (not shown) to determine which areas of the genome have unacceptably low coverage and then exclude them from the genome, thereby helping to ensure that when a new test sample is compared to (or using) the generated core genome, the portions of the core genome are likely to be present in the new test sample. In some embodiments, low coverage portions may be removed from consideration before high conservation portions are selected, while in others, the low coverage portions may be removed from the core genome after the high conservation portions are selected. As yet another alternative, the two operations may be performed in parallel or otherwise independent from each other to generate a set of highly conserved locations and a set of high coverage locations; a unions of the two sets may then produce the desired core genome. Various other algorithmic structures may be apparent.

To select high coverage areas, the method may obtain a set of samples and align them against a reference genome (e.g., the reference selected in step 120). Next, a tool such as mpileup may be used to compute coverage values for each position of each sample in the set. These values may then be combined to produce an average (or median or other statistical metric) coverage for each position in the genome. Thereafter, a threshold may be applied to each position's average coverage metric to determine whether that position is a high coverage position. For example, the average coverage may be compared to an absolute cutoff (e.g., position found in 20 reads or more) or a relative cutoff (e.g. position found in 20% of reads or greater).

It is interesting to note that coverage statistics are highly dependent on the sequencing technology being used and, as such, a core genome constructed in this manner to exclude low coverage areas would be primarily useful for the same sequencing technology from which the set of samples is obtained. For example, if the core genome is created based on location that are highly covered in a set of samples from a short read sequencer, such core genome may not be optimal for use with new samples obtained from a long read nanopore sequencer. Thus, if a core genome is needed for samples of a new sequencing technology, the process (or at least the portion of the process that identifies high coverage locations or depends thereon) would be repeated.

At step 170 of the method, the genome reference system assigns the selected base positions to a genome reference. The genome reference will comprise a plurality of selected base positions, and may comprise one region of the species' genome, multiple regions of the species' genome, or the entire genome. For example, the genome reference may comprise only base positions that exceed the predetermined threshold, or may comprise both base positions that exceed the predetermined threshold and base positions that do not.

According to one embodiment, the generated genome reference can be combined with a traditional core genome by taking the intersection or union of both reference bases. For example, a combined genome reference may comprise only those regions that agree between a generated genome reference and a traditional reference genome including but not limited to a core genome.

According to an embodiment, the base positions in the generated genome reference, or the base positions utilized for the generated genome reference, may be undergo filtering based on one more criteria. For example, the base positions assigned to the genome reference may be filtered using known biological information to make the genomic comparisons more meaningful to physicians and infectious disease specialists. Many other filters are possible.

At step 180 of the method, the genome reference system stores the generated genome reference in a data structure. According to an embodiment, the selected base positions are associated with a genome reference identifier in a data structure, such as a table or other structure in memory, a database, or other storage means. In addition to being associated with the genome reference in a data structure, each of the selected base positions may also comprise the determined frequency information for that base position.

At step 190 of the method, the genome reference system compares a new sample genome from the species to the generated genome reference. For example, the genome reference system may align the sequencing data from the new sample genome with the generated genome reference to determine and calculate similarity between the new sample genome and the generated genome reference. The alignment and similarity may be performed, for example, using known methods of alignment and similarity determination. According to an embodiment, samples can be compared against the generated genome reference by considering only the base positions found within the generated genome reference when calculating genomic distance between the two genomes.

As a result of the claimed method and system, the genomic distances calculated using the generated genome reference exhibit far greater stability and reproducibility, and are more suitable for standardized audit trails. Indeed, in trials of the claimed method and system on tests using several pathogens (K. pneumoniae, S. aureus, P. aeruginosa), a genomic basis constructed with NCBI RefSeq reference genomes according to this method led to increased resolution between genomically closely related and unrelated pathogens than a core genome approach.

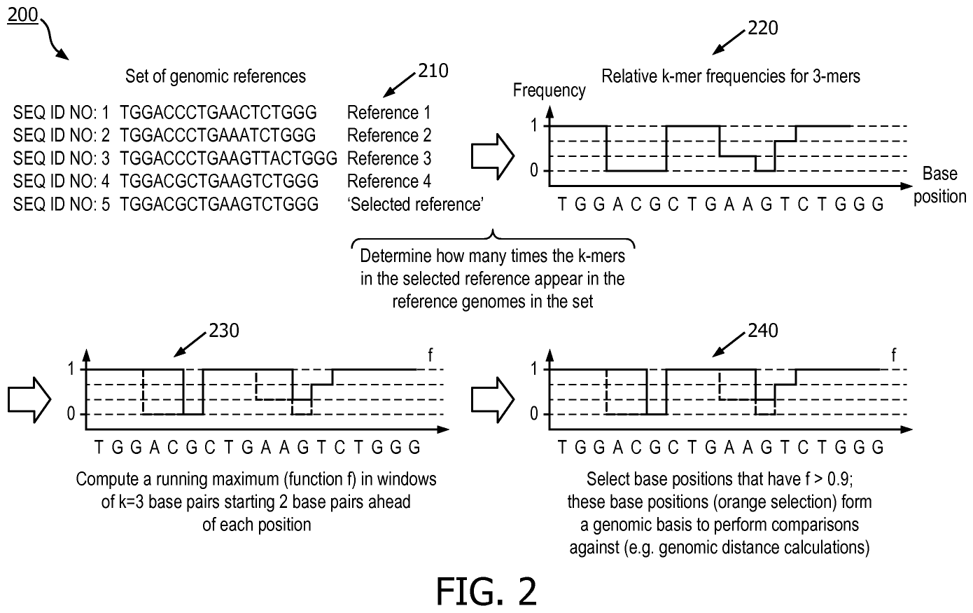

Referring to FIG. 2, in one embodiment, is a flowchart of a method 200 for generating a genome reference using a genome reference system. The genome reference system may be any of the systems described or otherwise envisioned herein, and may comprise any of the components described or otherwise envisioned herein.

At step 210 of the method, a genome reference system comprises a set of genomic references for a species. As described or otherwise envisioned herein, the set of genomic references may be generated or received. Also at step 210 of the method, one of the genomic references within the set of genomic references is chosen as a selected reference.

At step 220 of the method, the genome reference system determines how many times the k-mers in the selected reference appear in the reference genomes in the set, thereby determining a frequency for each of the k-mers. According to an embodiment, the genome reference system aligns the k-mers with the reference genomes in the set. Although FIG. 2 shows the k-mers as 3-mers, this is a non-limiting example and the k-mers can be of any length.

At step 230 of the method, the genome reference system computes a running maximum, average, or other function in windows of k=3 base pairs, for the described 3-mers, starting 2 base pairs ahead of each base position. The transformation function will be adapted based on, for example, the length of the k-mers in the data set and/or at this region.

At step 240 of the method, the genome reference system selects base positions that meet a predetermined threshold. For example, referring to FIG. 2, the genome reference system selects base positions that have a frequency (f)>0.9. These selected base positions form a genome basis, a genome reference, against which new samples can be compared to determine genetic distances.

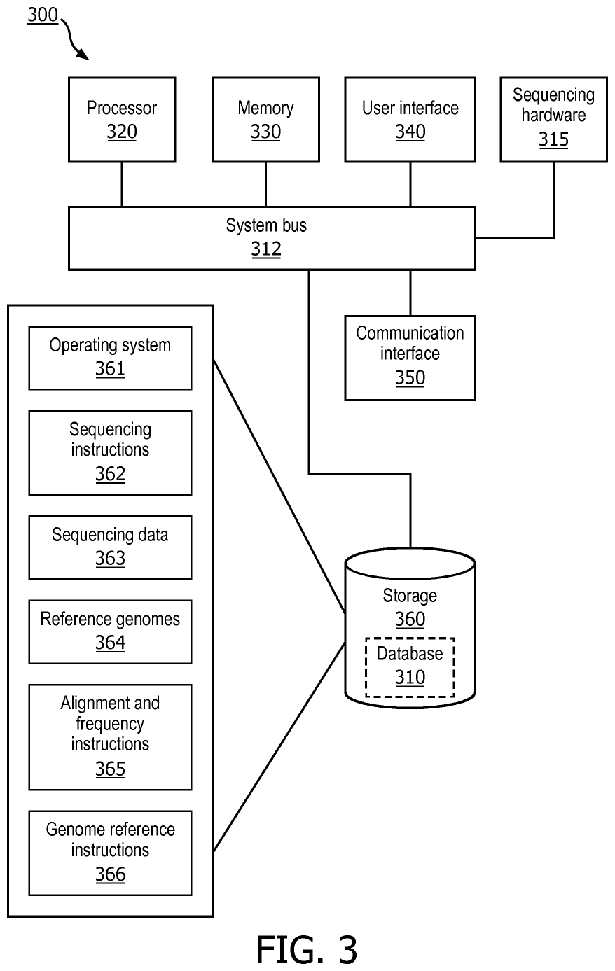

Referring to FIG. 3, in one embodiment, is a schematic representation of a genome reference system 300 for generating a genome reference. System 300 may be any of the systems described or otherwise envisioned herein, and may comprise any of the components described or otherwise envisioned herein.

According to an embodiment, system 300 comprises one or more of a processor 320, memory 330, user interface 340, communications interface 350, and storage 360, interconnected via one or more system buses 312. In some embodiments, such as those where the system comprises or directly implements a sequencer or sequencing platform, the hardware may include additional sequencing hardware 315 such as a real-time single-molecule sequencer, including but not limited to a pore-based sequencer, although many other sequencing platforms are possible. It will be understood that FIG. 3 constitutes, in some respects, an abstraction and that the actual organization of the components of the system 300 may be different and more complex than illustrated.

According to an embodiment, system 300 comprises a processor 320 capable of executing instructions stored in memory 330 or storage 360 or otherwise processing data to, for example, perform one or more steps of the method. Processor 320 may be formed of one or multiple modules. Processor 320 may take any suitable form, including but not limited to a microprocessor, microcontroller, multiple microcontrollers, circuitry, field programmable gate array (FPGA), application-specific integrated circuit (ASIC), a single processor, or plural processors.

Memory 330 can take any suitable form, including a non-volatile memory and/or RAM. The memory 330 may include various memories such as, for example L1, L2, or L3 cache or system memory. As such, the memory 330 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 300. It will be apparent that, in embodiments where the processor implements one or more of the functions described herein in hardware, the software described as corresponding to such functionality in other embodiments may be omitted.

User interface 340 may include one or more devices for enabling communication with a user. The user interface can be any device or system that allows information to be conveyed and/or received, and may include a display, a mouse, and/or a keyboard for receiving user commands. In some embodiments, user interface 340 may include a command line interface or graphical user interface that may be presented to a remote terminal via communication interface 350. The user interface may be located with one or more other components of the system, or may located remote from the system and in communication via a wired and/or wireless communications network.

Communication interface 350 may include one or more devices for enabling communication with other hardware devices. For example, communication interface 350 may include a network interface card (NIC) configured to communicate according to the Ethernet protocol. Additionally, communication interface 350 may implement a TCP/IP stack for communication according to the TCP/IP protocols. Various alternative or additional hardware or configurations for communication interface 350 will be apparent.

Storage 360 may include one or more machine-readable storage media such as read-only memory (ROM), random-access memory (RAM), magnetic disk storage media, optical storage media, flash-memory devices, or similar storage media. In various embodiments, storage 360 may store instructions for execution by processor 320 or data upon which processor 320 may operate. For example, storage 360 may store an operating system 361 for controlling various operations of system 300. Where system 300 implements a sequencer and includes sequencing hardware 315, storage 360 may include sequencing instructions 362 for operating the sequencing hardware 315, and sequencing data 363 obtained by the sequencing hardware 315. Storage 360 may also store one or more reference genomes 364.

It will be apparent that various information described as stored in storage 360 may be additionally or alternatively stored in memory 330. In this respect, memory 330 may also be considered to constitute a storage device and storage 360 may be considered a memory. Various other arrangements will be apparent. Further, memory 330 and storage 360 may both be considered to be non-transitory machine-readable media. As used herein, the term non-transitory will be understood to exclude transitory signals but to include all forms of storage, including both volatile and non-volatile memories.

According to an embodiment, system 300 comprises or is in communication with a reference genome database 310. The reference genome database may be a local database or a remote database, a public database or a private database. For example, as shown in FIG. 3, the reference genome database 310 may be stored in storage 360. As another example, the reference genome database 310 may be stored remotely and accessed via the communication interface. The reference genome database 310 may comprise one or more reference genomes, including the sequencing data associated with one of more of the reference genomes.

While genome reference system 300 is shown as including one of each described component, the various components may be duplicated in various embodiments. For example, processor 320 may include multiple microprocessors that are configured to independently execute the methods described herein or are configured to perform steps or subroutines of the methods described herein such that the multiple processors cooperate to achieve the functionality described herein. Further, where one or more components of system 300 is implemented in a cloud computing system, the various hardware components may belong to separate physical systems. For example, processor 320 may include a first processor in a first server and a second processor in a second server. Many other variations and configurations are possible.

According to an embodiment, storage 360 of genome reference system 300 may store one or more algorithms and/or instructions to carry out one or more functions or steps of the methods described or otherwise envisioned herein. For example, processor 320 may comprise alignment and frequency instructions 365, and/or genome reference instructions 366.

According to an embodiment, alignment and frequency algorithm or instructions 365 direct the system to align the sequencing data from a selected reference against one or more reference genomes from a species, and to calculate the frequency of that sequencing data among the one or more reference genomes. For example, according to an embodiment, the genome reference system generates and/or receives sequencing data for a plurality of genomes. The genome reference system may comprise a sequencing platform configured to obtain one or more genomes for the plurality of genomes, or may receive one or more genomes for the plurality of genomes from a database or other source.

According to an embodiment, the alignment and frequency instructions 365 direct the system to select one of the plurality of genomes received or generated by the genome reference system to be a selected reference. The selected reference can be any of the genomes received or generated by the genome reference system.

According to an embodiment, the alignment and frequency instructions 365 direct the system to align the sequencing data from the selected reference with the remainder of the genomes in the plurality of genomes. For example, the sequencing data for the selected genome may comprise a plurality of k-mers that are aligned with each of the other genomes for the species in the database or otherwise obtained or generated by the genome reference system. The sequencing data from the selected reference may be aligned with the remainder of the genomes using any method of alignment, including but not limited to known alignment algorithms or methods.

According to an embodiment, the alignment and frequency instructions 365 direct the system to use the alignment information to determine a frequency of the sequencing data within the plurality of genomes. The alignment and frequency instructions 365 direct the system to track or record the alignment frequency for each piece of sequencing data for the selected reference, such as a k-mer, for example using a counter or any other tracking or recording method. Thus the alignment and frequency instructions 365 direct the system to generate and comprise an identification of alignment frequency for the sequencing data, such as for the plurality of k-mers.

According to an embodiment, the alignment and frequency instructions 365 direct the system to identify one or more base positions with the sequencing data using a transformation function. To measure the frequency of a single base within the sequencing data, which may comprise overlapping k-mers or other overlapping sequencing data, a transformation function is applied to the data. For example, the system may perform a running maximum, average, or another function of the relative counts as a frequency measure, among other transformation function.

According to an embodiment, the genome reference algorithm or instructions 366 direct the system to select base positions of the selected reference that meet or exceed a predetermined frequency threshold, and assigns them to a genome reference that is then stored and utilized for calculating genomic distances for new samples.

According to an embodiment, the genome reference instructions 366 direct the system to select one or more base positions of the selected reference that exceeds a predetermined frequency threshold. For example, each of the base positions of the selected reference may be associated with a frequency determined in one or more of the previous steps of the method. According to an embodiment, all base positions and/or sequencing data that exceed the predetermined threshold may be selected. As another option, only some base positions and/or sequencing data that exceed the predetermined threshold may be selected.

According to an embodiment, the genome reference instructions 366 direct the system to assign the selected base positions to a genome reference. The genome reference will comprise a plurality of selected base positions, and may comprise one region of the species' genome, multiple regions of the species' genome, or the entire genome. For example, the genome reference may comprise only base positions that exceed the predetermined threshold, or may comprise both base positions that exceed the predetermined threshold and base positions that do not.

According to an embodiment, the genome reference instructions 366 direct the system to store the generated genome reference in a data structure. According to an embodiment, the selected base positions are associated with a genome reference identifier in a data structure, such as a table or other structure in memory, a database, or other storage means. In addition to being associated with the genome reference in a data structure, each of the selected base positions may also comprise the determined frequency information for that base position.

According to an embodiment, the genome reference instructions 366 direct the system to compare a new sample genome to the generated genome reference to calculate similarity between the new sample genome and the generated genome reference.

The reference genome approach described or otherwise envisioned herein provides numerous advantages over existing systems. For example, a generated genome reference can be used as a fixed core genome, produces consistent single nucleotide variant (SNV) distances, and performs better than current fixed core genome approaches. A generated genome reference maintains the ability to distinguish same-pathogen samples from different-pathogen samples, but can also be applied in prospective clinical studies in which samples are continuously added and analyzed, and which require a fixed core genome that is defined a priori and does not change throughout the study. This is often needed to make sure that sample SNV distances do not change throughout the study, such that the SNV distance between samples A and B does not depend on sample C, for example. In this way, the interpretation is consistent and the clinician can make significantly improved decisions.

The current system also improves the functionality of the system as it results in the system being significantly more computationally more efficient, since sample distances do not have to be recomputed. Instead, only distances with the newly added samples need to be computed. Further, the k-mer analysis described herein generates a very quick conservation score for each nucleotide in the reference genome, compared to traditional core genome approaches.

Studies using the k-mer based conserved-nucleotide genome reference described herein have found that this approach is better than traditional approaches, such as the conserved-gene core genome, at distinguishing same-pathogen samples from different-pathogen samples. The genome reference approach described herein yields better true positive rates relative to false positive rates, where the true positives are correctly identified same-patient samples which are highly likely to be same-pathogen samples. Indeed, the approach described herein outperformed the traditional approach or approaches on every pathogen that was tested.

The core genome consists of highly conserved regions, whether they are gene regions or not. There are other ways to compute conservation scores for each nucleotide in the reference genome but these would typically be quite slow, e.g. multi-sequence alignment, whereas the approach described herein is very fast. The transformation to go from k-mer frequencies to nucleotide frequencies is non-trivial.

Furthermore, the genome reference approach described herein simplifies the creation of new genome references for new organisms, as it does not require, for example, gene annotation.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/of” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for generating a genome reference of a pathogenic species using a genome reference system, comprising:
receiving, by the system, sequencing data for a plurality of genomes, the sequencing data generated from a plurality of genomes obtained from a single pathogenic species;
selecting, by a processor of the system, sequencing data from one of the plurality of genomes;
aligning, by the processor, the selected sequencing data from the selected genome, comprising a plurality of k-mers, with each of the plurality of genomes;
determining, by the processor, based on the alignment, a frequency of each of the plurality of k-mers within the plurality of genomes;
selecting, by the processor based on the frequency determination, one or more base positions within the plurality of k-mers, wherein the one or more base positions exceed a predetermined frequency threshold;
assigning, by the processor, the selected base positions to a genome reference; and
storing the genome reference in a data structure.

2. The method of claim 1, wherein the sequencing data comprises whole genome sequencing data.

3. The method of claim 1, wherein the sequencing data comprises genome assemblies.

4. The method of claim 1, further comprising the step of identifying base positions within the plurality of k-mers by applying a transformation function to the sequencing data.

5. The method of claim 3, wherein the transformation function is a running maximum or a running average.

6. The method of claim 1, wherein the step of aligning the selected sequencing data from the selected genome with each of the plurality of genomes requires identity between the sequencing data and a region of the one of the plurality of genomes.

7. The method of claim 1, wherein the step of aligning the selected sequencing data from the selected genome with each of the plurality of genomes allows a predetermined level of mismatch between the sequencing data and a region of the one of the plurality of genomes.

8. The method of claim 1, further comprising the step of comparing a sample from a pathogen to the genome reference.

9. The method of claim 1, wherein receiving sequencing data for a plurality of genomes comprises generating sequencing data using a sequencing platform.

10. The method of claim 1, further comprising:
computing coverage metrics for a plurality of base positions across a plurality of sequence samples obtained from a single species; and
comparing the coverage metrics for the plurality of base positions to a predetermined coverage threshold to identify a set of highly covered base positions,
wherein selecting the one or more base positions comprises selecting one or more base positions within the plurality of k-mers that both:
exceed the predetermined frequency threshold, and
are associated with a coverage metric of the coverage metrics that exceed the predetermined coverage threshold.

11. A system for generating a genome reference of a pathogen species using a genome reference system, the system comprising:
a processor configured to: (i) receive sequencing data for a plurality of genomes, the sequencing data generated from a plurality of genomes obtained from a single pathogen species; (ii) select sequencing data from one of the plurality of genomes; (iii) align the selected sequencing data from the selected genome, comprising a plurality of k-mers, with each of the plurality of genomes; (iv) determined, based on the alignment, a frequency of each of the plurality of k-mers within the plurality of genomes; (v) select, based on the frequency determination, one or more base positions within the plurality of k-mers that exceed a predetermined frequency threshold; and (vi) assign the selected base positions to a genome reference; and
a data structure configured to store the genome reference.

12. The system of claim 11, wherein the processor is further configured to identify base positions within the plurality of k-mers using a transformation function.

13. The system of claim 12, wherein the transformation function is a running maximum or a running average.

14. The system of claim 11, wherein aligning the selected sequencing data from the selected genome with each of the plurality of genomes requires identity between the sequencing data and a region of the one of the plurality of genomes.

15. The system of claim 11, wherein aligning the selected sequencing data from the selected genome with each of the plurality of genomes allows a predetermined level of mismatch between the sequencing data and a region of the one of the plurality of genomes.

16. The system of claim 11, wherein the processor is further configured to compare a sample from a pathogen to the genome reference.

17. The system of claim 11, wherein the processor is further configured to:
compute coverage metrics for a plurality of base positions across a plurality of sequence samples obtained from a single species; and
compare the coverage metrics for the plurality of base positions to a predetermined coverage threshold to identify a set of highly covered base positions,
wherein, in selecting the one or more base positions, the processor is configured to select one or more base positions within the plurality of k-mers that both:
exceed the predetermined frequency threshold, and
are associated with a coverage metric of the coverage metrics that exceed the predetermined coverage threshold.


SEQUENCE LISTING (5 sequences)

SEQ ID NO: 1 - DNA, 18 bp, Artificial Sequence
  Artificial sequence generated as an example
tggaccctgaactctggg

SEQ ID NO: 2 - DNA, 18 bp, Artificial Sequence
  Artificial sequence generated as an example
tggaccctgaaatctggg

SEQ ID NO: 3 - DNA, 20 bp, Artificial Sequence
  Artificial sequence generated as an example
tggaccctgaagttactggg

SEQ ID NO: 4 - DNA, 18 bp, Artificial Sequence
  Artificial sequence generated as an example
tggacgctgaagtctggg

SEQ ID NO: 5 - DNA, 18 bp, Artificial Sequence
  Artificial sequence generated as an example
tggacgctgaagtctggg